(19) United States
(12) Patent Application Publication
(10) Pub. No.: US 2024/0082286 A1
(43) Pub. Date: Mar. 14, 2024

(54) COMBINATION THERAPIES FOR TREATMENT OF T-CELL LYMPHOMAS

(71) Applicant: Otsuka Pharmaceutical Co., Ltd., Tokyo (JP)

(72) Inventors: Martin John Sims, Waresley (GB); George Albert Ward, Bury St. Edmunds (GB); Harold Keer, Emerald Hills (CA); Aram Oganesian, Dublin (CA)

(21) Appl. No.: 18/458,884

(22) Filed: Aug. 30, 2023

Related U.S. Application Data
(60) Provisional application No. 63/374,137, filed on Aug. 31, 2022.

(30) Foreign Application Priority Data
Nov. 4, 2022 (WO) PCT/IB2022/060652

(51) Int. Cl. A61K 31/706; A61K 31/5377; A61P 35/00

(52) U.S. Cl. CPC A61K 31/706; A61K 31/5377; A61P 35/00

(57) ABSTRACT

The present disclosure relates generally to methods of treating T-cell lymphomas with combination therapies.

### CROSS REFERENCE TO RELATED APPLICATIONS

This application claims priority to International Application No. PCT/IB2022/060652, filed Nov. 4, 2022, which claims priority to U.S. Provisional Application No. 63/374,137 filed Aug. 31, 2022, each of which are hereby incorporated by reference herein in their entireties.

### FIELD

The present disclosure is directed to treatment of T-cell lymphoma with certain combination therapies.

### BACKGROUND

Evasion of apoptosis is one of the hallmarks of cancer and apoptosis is a mechanism for programmed cell death that is dysregulated in many tumor types. Inhibitors of apoptosis proteins (IAPs) are key regulators of antiapoptotic and pro-survival signaling pathways; they are often overexpressed in cancer cells and associated with tumor progression and resistance to treatment. Sometimes, T-cell lymphomas acquire resistance to IAP inhibitor therapies. There is a need for improved treatments for T-cell lymphoma, including therapies that can re-sensitize tumor cells, that are otherwise resistant to the effects of IAPs, to IAP inhibitors.

### SUMMARY

It is contemplated that combining tolinapant with decitabine enhances immunogenic cell death in T-cell lymphoma. It is further contemplated that the decitabine may be administered orally when in combination with cedazuridine. The present disclosure, in one embodiment, provides a method for treating a T-cell lymphoma in a subject in need thereof comprising administering to the subject
tolinapant, or a pharmaceutically acceptable salt thereof;
cedazuridine, or a pharmaceutically acceptable salt thereof; and
decitabine, or a pharmaceutically acceptable salt thereof.

In one embodiment, the administration of each agent is oral.

Also provided is a pharmaceutical composition comprising tolinapant, or a pharmaceutically acceptable salt thereof, cedazuridine, or a pharmaceutically acceptable salt thereof, and decitabine, or a pharmaceutically acceptable salt thereof.

An advantage of the combination therapy described herein is that tolinapant, cedazuridine, and decitabine, are administered orally, which can potentially improve patient compliance.

### BRIEF DESCRIPTION OF THE DRAWINGS

FIG. 1 is a photograph of western-blot results demonstrating RIPK3 expression in CT26 cells (a mouse colon adenocarcinoma cell line), either parental or genetically manipulated to express RIPK3 (A), and a graph showing lytic cell death upon treatment of tolinapant over-time (B), according to an embodiment.

FIG. 2 is a photograph of western-blot results of RIPK3 expression in human and mouse cell lines including T-cell lymphoma cell lines according to an embodiment.

FIG. 3 is a graph illustrating the mean baseline methylation level of RIPK3 gene in different cell lines (A), a graph illustrating relative change in methylation level of RIPK3 gene in different cell lines after decitabine treatment (B), a graph illustrating dose response and time course of RIPK3 demethylation in Karpas-299 cells by decitabine treatment (C), and a graph showing relative change in methylation level of LINE-1 after decitabine treatment (D), according to an embodiment.

FIG. 4 is a graph illustrating the concentraction of IFNγ released from H9 cells after being treated with decitabine (DAC) in different concentrations, according to one embodiment.

FIG. 5 illustrates graphs showing the reduction of cell viability and level of synergy by the combination of decitabine and tolinapant according to one embodiment.

FIG. 6 illustrates a graph showing lytic cell death of human H9 cells over time upon treatment with tolinapant and/or decitabine according to one embodiment.

FIG. 7 illustrates a graph showing HMGB1 (a biomarker of lytic cell death) concentration upon treatment with tolinapant and/or decitabine according to one embodiment.

FIG. 8 is a photograph of western-blot result of BW5147.G.1.4 cell line lysates after 48 hour treatment with decitabine and tolinapant, according to one embodiment.

FIG. 9 illustrates graphs A and B showing concentration of cytokines and chemokines secreted in vitro after treatment of BW5147 cells with tolinapant and/or decitabine for 48 hours according to one embodiment.

FIG. 10 is a photograph of western-blot results of BW5147.G.1.4 cell line lysates after 5 day treatment with decitabine and/or tolinapant in vivo (A) and a graph showing the plasma HMGB1 concentration after 5 day treatment with decitabine and/or tolinapant in vivo (B), according to one embodiment.

FIG. 11 illustrates graphs showing plasma concentration of cytokines and chemokines after treatment of BW5147.G.1.4 cells in vivo with tolinapant and/or decitabine for 5 days according to one embodiment.

FIG. 12 illustrates relative expression level of several genes in KARPAS-299 xenografts after treatment with tolinapant and/or decitabine according to one embodiment.

FIG. 13 is a photograph of western-blot results of KARPAS-299 xenograft cell lysates after treatment with decitabine or tolinapant and decitabine according to one embodiment.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

A dash (“—”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of tolinapant, cedazuridine, and/or decitabine, in which from 1 to n hydrogens attached to a carbon atom is/are replaced by deuterium, in which n is the number of hydrogens in the molecule. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure, i.e., tolinapant, cedazuridine, and/or decitabine, are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

Tolinapant is described in U.S. Pat. No. 9,783,538 and has a structure as follows:

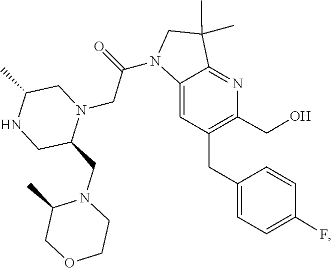 [SMILES: C[C@@H]1CN(CC(=O)N2CC(C)(C)c3nc(CO)c(Cc4ccc(F)cc4)cc32)[C@@H](CN2CCOC[C@H]2C)CN1]

and is named 1-(6-(4-fluorobenzyl)-5-(hydroxymethyl)-3,3-dimethyl-2,3-dihydro-1H-pyrrolo[3,2-b]pyridin-1-yl)-2-((2R,5R)-5-methyl-2-(((R)-3-methylmorpholino)methyl)piperazin-1-yl)ethan-1-one (or alternatively 1-{6-[(4-fluorophenyl)methyl]-5-(hydroxymethyl)-3,3-dimethyl-1H,2H,3H-pyrrolo[3,2-b]pyridin-1-yl}-2-[(2R,5R)-5-methyl-2-{[(3R)-3-methylmorpholin-4-yl]methyl}piperazin-1-yl]ethan-1-one).

Cedazuridine is described in U.S. Pat. No. 8,268,800 and has a structure as follows:

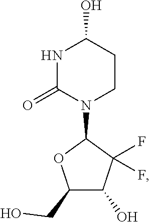 [SMILES: O=C1N[C@H](O)CCN1[C@@H]1O[C@H](CO)[C@@H](O)C1(F)F]

and is named (R)-1-((2R,4R,5R)-3,3-difluoro-4-hydroxy-5-(hydroxymethyl)tetrahydrofuran-2-yl)-4-hydroxytetrahydropyrimidin-2(1H)-one

Decitabine has a structure follows:

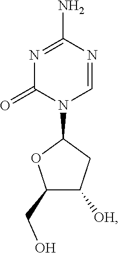 [SMILES: Nc1ncn([C@H]2C[C@H](O)[C@@H](CO)O2)c(=O)n1]

and is named 5-aza-2′-deoxycytidine or 4-amino-1-((2R,4S,5R)-4-hydroxy-5-(hydroxymethyl)tetrahydrofuran-2-yl)-1,3,5-triazin-2(1H)-one.

### Treatment Methods and Uses

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of T-cell lymphoma. The therapeutically effective amount may vary depending on the subject, and disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

In some embodiments, provided herein is a method for treating a T-cell lymphoma in a subject in need thereof comprising administering to the subject
tolinapant, or a pharmaceutically acceptable salt thereof;
cedazuridine, or a pharmaceutically acceptable salt thereof; and
decitabine, or a pharmaceutically acceptable salt thereof.

In some embodiments, the T-cell lymphoma is relapsed or refractory T-cell lymphoma. In some embodiments, the T-cell lymphoma was previously treated with a hypomethylating agent. In some embodiments, the hypomethylating agent is decitabine. In some embodiments, the T-cell lymphoma has not been previously treated with an agent for treating T-cell lymphoma.

In some embodiments, the T-cell lymphoma is selected from peripheral T-cell lymphomas, peripheral T-cell lymphomas not otherwise specified, angioimmunoblastic T-cell lymphoma, follicular T-cell lymphoma, nodal peripheral T-cell with T-follicular helper (THF) phenotype, adult T-cell lymphoma/leukemia, anaplastic large cell lymphoma, enteropathy-associated T-cell lymphoma, nasal NK/T-cell lymphoma, hepatosplenic T-cell lymphoma, monomorphic epitheliotropic intestinal T-cell lymphoma, subcutaneous panniculitis-like T cell lymphoma, and cutaneous (skin) T-cell lymphoma.

In some embodiments, the T-cell lymphoma is relapsed or refractory peripheral T-cell lymphoma (R/R PTCL).

In some embodiments, the T-cell lymphoma is peripheral T-cell lymphoma not otherwise specified. In some embodiments, the T-cell lymphoma is angioimmunoblastic lymphoma.

In some embodiments, the T-cell lymphoma is nodal peripheral T-cell with T-follicular helper (THF) phenotype. In some embodiments, the T-cell lymphoma is adult T-cell lymphoma/leukemia,

In some embodiments, the T-cell lymphoma is anaplastic large cell lymphoma (ALCL).

In some embodiments, the T-cell lymphoma is hepatosplenic T-cell lymphoma. In some embodiments, the T-cell lymphoma is monomorphic epitheliotropic intestinal T-cell lymphoma. In some embodiments, the T-cell lymphoma is subcutaneous panniculitis-like T cell lymphoma. In some embodiments, the T-cell lymphoma is cutaneous (skin) T-cell lymphoma.

In some embodiments, the T-cell lymphoma is enteropathy-associated T-cell lymphoma.

In some embodiments, the T-cell lymphoma is cutaneous T-cell lymphoma. In some embodiments, the cutaneous T-cell lymphoma is mycosis fungoides or Sézary syndrome.

In some embodiments, the T-cell lymphoma is T-lymphoblastic lymphoma/leukemia or adult T-cell lymphoma/leukemia.

In some embodiments, the tolinapant is administered once a day for 7 consecutive days every other week of each 28-day cycle. In some embodiments, the dose of tolinapant is 30 mg or 90 mg daily.

In some embodiments, the cedazuridine and the decitabine are administered once daily on days 1 through 5 of each 28-day cycle. In some embodiments, the dose of cedazuridine is 100 mg daily and the dose of decitabine is 35 mg daily.

In some embodiments, the subject is not being treated with any compound that is metabolized by cytidine deaminase.

In some embodiments, tolinapant, cedazuridine, and decitabine are administered orally.

Additional Combination Therapies

In one embodiment, the compounds disclosed herein may be used in combination with one or more additional therapeutic agents that are being used and/or developed to treat T-cell lymphomas, such as bone marrow/stem cell transplant and/or CAR T cell therapies.

In some embodiments, the one or more additional therapeutic agent may be an additional IAP inhibitor.

Kits

Provided herein are also kits that include a tolinapant, and/or cedazuridine, and/or decitabine, or a pharmaceutically acceptable salt, tautomer, stereoisomer, mixture of stereoisomers, prodrug, or deuterated analog of each, and suitable packaging. In one embodiment, a kit further includes instructions for use. In one aspect, a kit includes a tolinapant, and/or cedazuridine, and/or decitabine, or a pharmaceutically acceptable salt, tautomer, stereoisomer, mixture of stereoisomers, prodrug, or deuterated analog of each, and a label and/or instructions for use of the compounds in the treatment of the indications, including the diseases or conditions, described herein.

Provided herein are also articles of manufacture that include any compound described herein or a pharmaceutically acceptable salt, tautomer, stereoisomer, mixture of stereoisomers, prodrug, or deuterated analog thereof in a suitable container. The container may be a vial, jar, ampoule, preloaded syringe, and intravenous bag.

Pharmaceutical Compositions and Modes of Administration

Compounds provided herein are usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In some embodiments, provided herein is a pharmaceutical composition comprising
tolinapant, or a pharmaceutically acceptable salt thereof;
a cedazuridine, or a pharmaceutically acceptable salt thereof; and
decitabine, or a pharmaceutically acceptable salt thereof.

The pharmaceutical compositions may be administered in either single or multiple doses. The pharmaceutical composition may be administered by various methods including, for example, by intra-arterial injection, intravenously, intraperitoneally, parenterally, intramuscularly, subcutaneously, or orally.

One mode for administration is parenteral, for example, by injection or intravenous drip. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, tautomer, stereoisomer, mixture of stereoisomers, prodrug, or deuterated analog thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In some embodiments, the cedazuridine, the decitabine, and the tolinapant are administered as individual tablets or capsules. In some embodiments, the cedazuridine and decitabine is administered as a tablet comprising a fixed dose combination of cedazuridine and decitabine. In some of such embodiments, the tablet comprises a fixed dose combination of 100 mg cedazuridine and 35 mg decitabine. In some of such embodiments, the tablet further comprises lactose monohydrate, hypromellose, croscarmellose sodium, colloidal silicon dioxide and magnesium stearate. In some embodiments, the tablet has a film coating comprising polyvinyl alcohol, titanium dioxide, polyethylene glycol, talc, and iron oxide red. In some embodiments, the tolinapant is administered as a capsule or a tablet in combination with a fixed dose combination tablet comprising cedazuridine and decitabine.

Dosing

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of tolinapant, cedazuridine and/or decitabine may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

In some embodiments, patients are administered tolinapant at 3 sequential dose levels (120, 150, and 180 mg/day on Days 1-7 and 15-21 of a 28-day cycle) in combination with fixed dose oral decitabine/cedazuridine or to a fixed dose of oral decitabine/cedazuridine as a single agent. The starting dosing of oral decitabine/cedazuridine in both arms will be the standard FDC tablet (35 mg decitabine/100 mg cedazuridine).

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Example 1-1

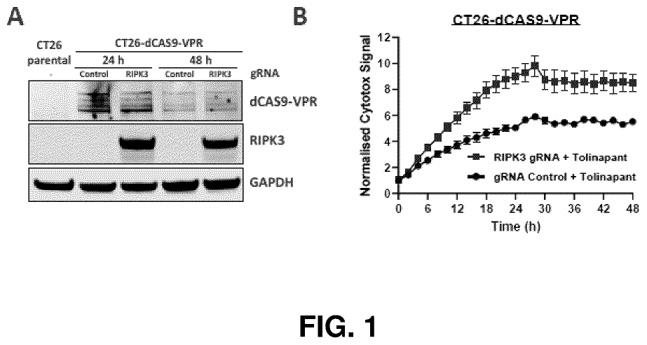

CT26 is a mouse colon adenocarcinoma cell line that does not express receptor-interacting serine/threonine-protein kinase 3 (RIPK3). CRISPR activation (CRISPRa) was used to re-express RIPK3 in the CT26 line (from ATCC) (FIG. 1A). CT26 control cells that do not express RIPK3 and the RIPK3 re-expressed CT26 cells were treated with 1 μM tolinapant. The lytic cell death upon treatment of tolinapant was measured for the CT26 control cells and the RIPK3 re-expressed CT26 cells, by real-time microscopy (IncuCyte) assays (FIG. 1B). As shown in FIG. 1B, the RIPK3 re-expressed CT26 cells exhibited higher lytic cell death upon treatment with tolinapant than the CT26 control cells, demonstrating that RIPK3 expression increased tolinapant-induced cell death.

### Example 1-2

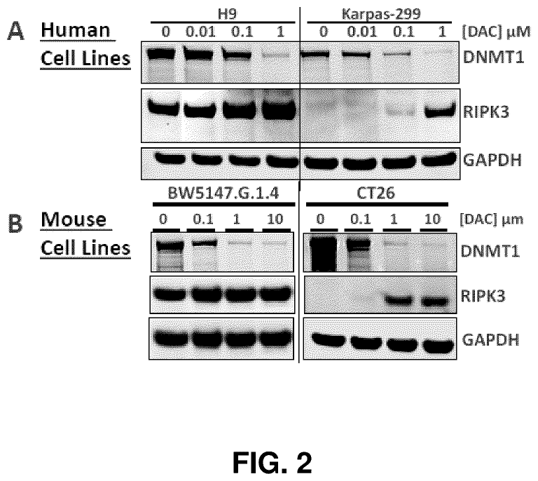

Two types of human T-cell lymphoma cell lines (H9, Karpas-299, from ECACC, Porton Down, Salisbury UK) were treated with 0.01, 0.1, 1 μM or no decitabine (DAC) for 4 days. Two types of mouse cell lines (BW5147.G.1.4, a T cell lymphoma from DSMZ (Braunschweig, Germany) and CT26) were treated with 0.01, 0.1, 1 μM or no decitabine (DAC) for 2 days. As shown in FIG. 2, RIPK3 expression was detected after the treatment with decitabine (DAC) of Karpas-299 or CT26 cells, even though RIPK3 is normally silenced in Karpas-299 or CT26 cells, showing that RIPK3 was re-expressed after the addition of decitabine, in both mouse and human cells. On the other hand, H9 cells and BW5147.G.1.4 exhibited high RIPK3 basal expression before treatment with decitabine (DAC), as shown in FIG. 2.

### Example 1-3

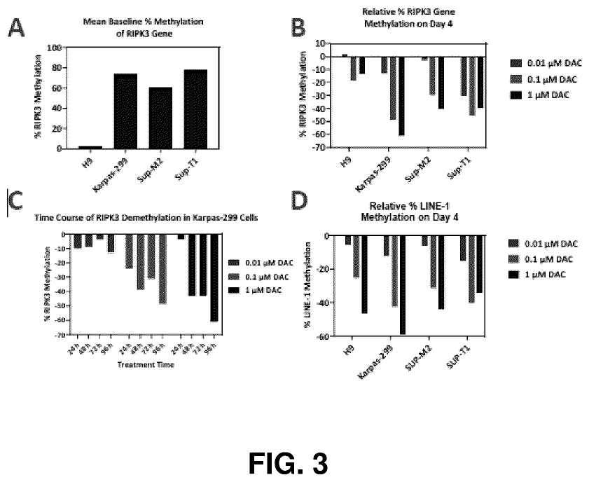

The DNA of human H9, Karpas-299, Sup-M2 (from DSMZ), and Sup-T1 (from ECACC) cell lines were sequenced to identify methylation status of the RIPK3 gene by pyrosequencing (EpigenDX). As shown in Panel A of FIG. 3, human H9 cells had low basal methylation of the RIPK3 gene promoter, while Karpas-299, Sup-M2, and Sup-T1 cells exhibited relatively high basal methylation of the RIPK3 gene promoter. These cell lines were treated with 0.01, 0.1, and 1 μM decitabine (DAC) for 4 days, and DNA of the cells were sequenced to identify methylation status of the RIPK3 gene by pyrosequencing (EpigenDX). As shown in Panels B and C of FIG. 3, decitabine (DAC) treatment led to demethylation of RIPK3 gene promoter overtime. Also, as shown in Panel D of FIG. 3, decitabine (DAC) treatment led to demethylation of LINE-1.

### Example 1-4

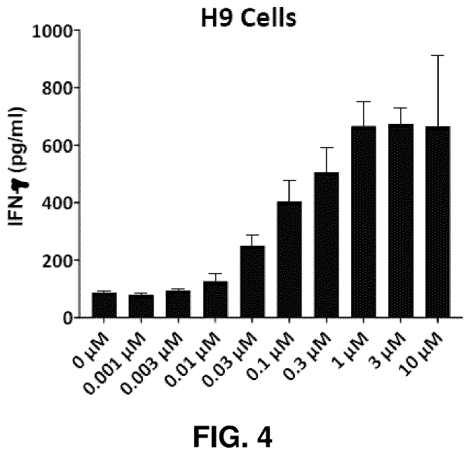

Human T-cell lymphoma (H9) cell line was treated with decitabine (DAC) (0.001, 0.003, 0.01, 0.03, 0.1, 0.3, 1, 3 and 10 μM) for 48 hours, and the release of IFNγ was measured an electrochemiluminescence immunoassay (Meso Scale Discovery). As shown in FIG. 4, IFNγ released from H9 cells were increased as the cells with the treatment of decitabine alone for 48 hours. The results from Examples 1-1 to 1-4 indicates that certain sensitive cells of T-cell lymphoma patients may re-express silenced promoters of members of the ripopotosome complex, such as RIPK3, upon treatment T-cell lymphoma patients, and the re-expressed repopotosome complex, including RIPK3, can increase tolinapant-induced cell death. Thus, the treatment by decitabine will potentially result in improved response rate to the tolinapant treatment.

### Example 1-5

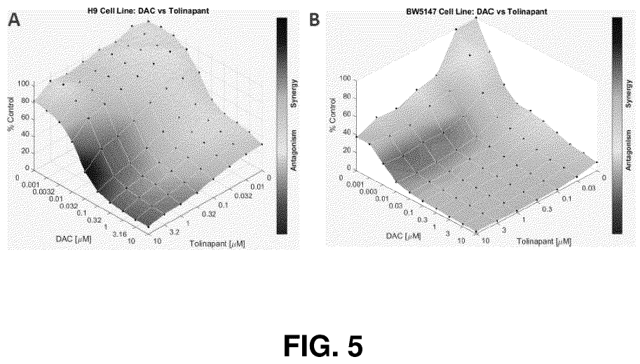

The human T-cell lymphoma cell line (H9) and mouse T-cell lymphoma cell line (BW5147.G.1.4) were treated with decitabine (DAC) only, tolinapant only, and the combination of decitabine (DAC) and tolinapant, for 72 hours, and cell viability was measured by a proliferation assay (CellTiterGlo). The combination of decitabine (DAC) and tolinapant reduced cell viability, and the synergy of decitabine (DAC) and tolinapant was assessed by HSA model using Combenenfit, where the plot is shown in FIG. 5 (Panel A: H9 cell line, Panel B: BW5147.G.1.4 cell line). As shown in FIG. 5, the combination of decitabine and tolinapant exhibited synergic effect, especially against the human H9 cell line.

### Example 1-6

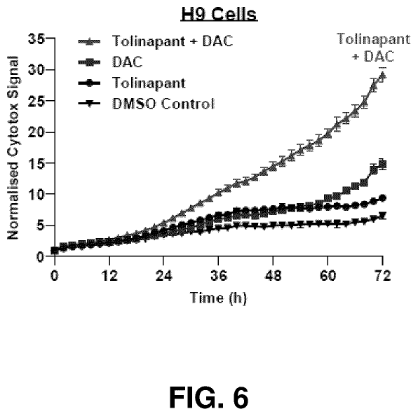

The human T-cell lymphoma (H9) cell line was treated with decitabine (DAC) only, tolinapant only, and the combination of decitabine (DAC) and tolinapant, for 72 hours, and the lytic cell death over-time, was measured by Cytotox staining of H9 cells by real-microscopy (IncuCyte) assays, and the result is shown in FIG. 6. As shown in FIG. 6, combination of decitabine (DAS) and tolinapant exhibited synergic increase of the lytic cell death.

### Example 1-7

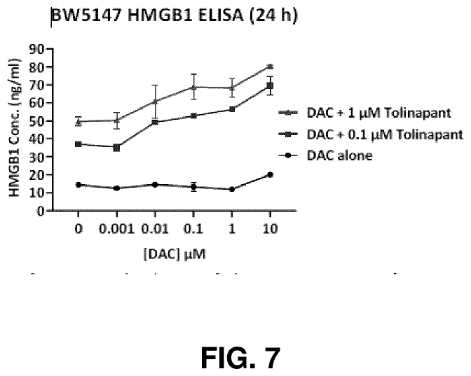

Mouse T-cell lymphoma (BW5147.G.1.4) cell line was treated with decitabine (DAC) only, (decitabine (DAC)+0.1 μM tolinapant), and (decitabine (DAC)+1 μM tolinapant) for 24 hours. The concentration of decitabine (DAC) was varied among 0, 0.001, 0.01, 0.1, 1, and 10 μM. HMGB1 released from the cells were measured by ELISA after 24 hours. The concentration of HMGB1 at different concentration of decitabine (DAC) and tolinapant is shown in FIG. 7. As shown in FIG. 7, the combination of decitabine (DAC) and tolinapant exhibited increased HMGB1 concentration than decitabine alone, indicating increased lytic cell death.

### Example 1-8

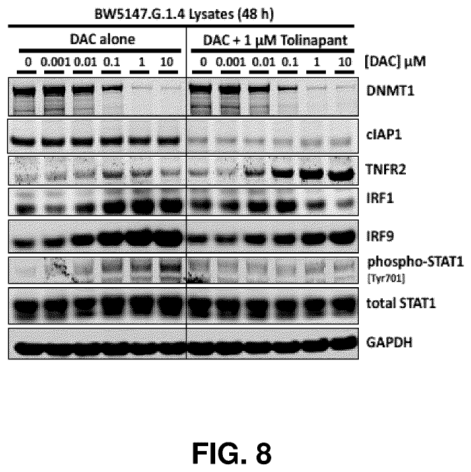

Mouse T-cell lymphoma (BW5147.G.1.4) cell line was treated with decitabine (DAC) only, or the combination of decitabine (DAC) and tolinapant for 48 hours. After 48 hours, the cell was lysated and interferon signaling and PD markers were analyzed by Western blotting, as shown in FIG. 8. From the western blot, it was confirmed that the expression of DNMT1 was reduced as the concentration of decitabine increased, and the expression of clAP1 was suppressed when tolinapant was added.

### Example 1-9

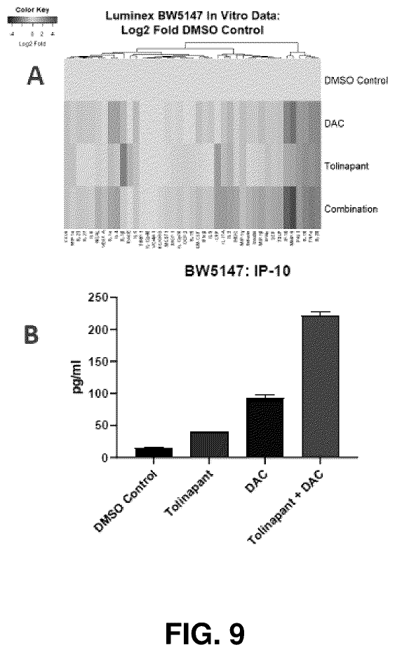

Mouse T-cell lymphoma (BW5147.G.1.4) cell line was treated with decitabine (DAC) only, tolinapant only, or the combination of decitabine (DAC) and tolinapant for 48 hours. After 48 hours, key inflammatory mediators were measured, as shown in Panel A of FIG. 9 by Luminex assay (Ampersand). As shown in Panel B of FIG. 9, the concentration of IP-10 were much higher when BW5147.G.1.4 cells were treated with the combination of decitabine and tolinapant, than decitabine or tolinapant alone.

### Example 1-10

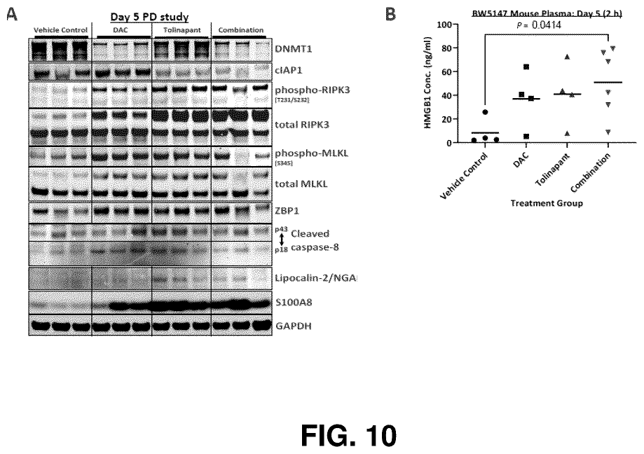

BW5147.G.1.4 tumor-bearing AKR/J mice were treated daily with decitabine (DAC) (0.3 mg/kg i.p.), tolinapant (25 mg/kg p.o.), or the combination thereof. The BW5147 cells were collected at day 5, and western blotting of the BW5147 cell lysates are shown in Panel A of FIG. 10.

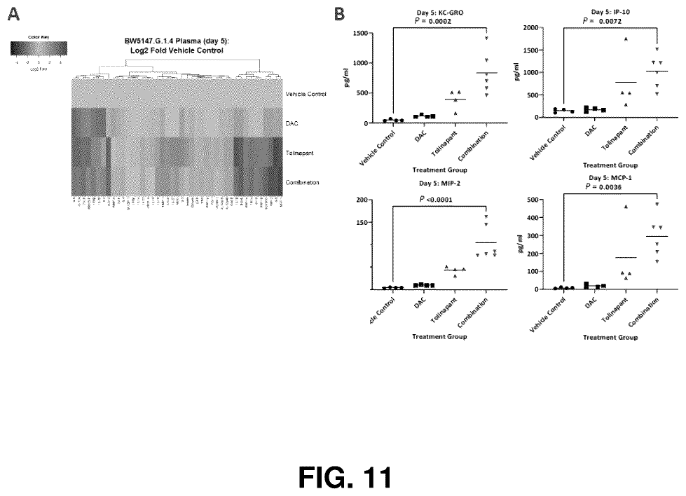

Plasma level of HMGB1 after dosing with decitabine (DAC), tolinapant and the combination for 5 days, was measured using ELISA, and the combination exhibited increased HMGB1 concentration, as shown in Panel B of FIG. 10. Cytokines and chemokines level in plasma after 5 days treatment was measured, and the result is shown in FIG. 11.

### Example 1-11

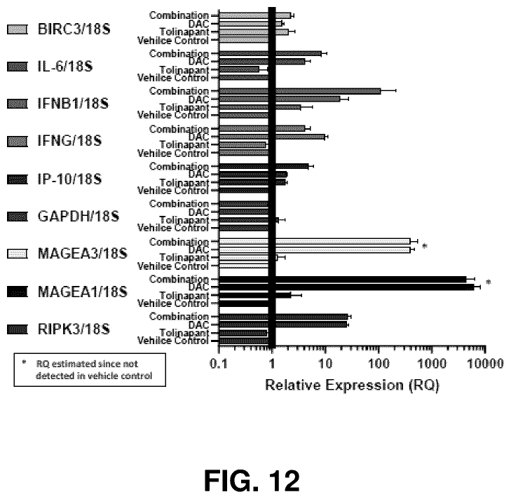

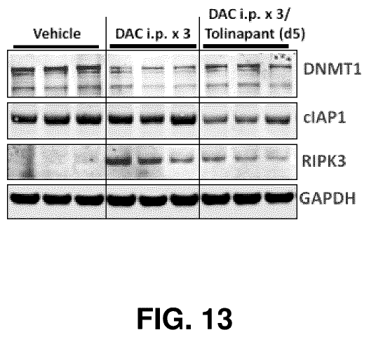

KARPAS-299 xenograft mouse model was prepared by injecting KARPAS-299 cells. The mice bearing the KARPAS-299 xenografts were treated with decitabine (DAC) and/or tolinapant for five days. The tumor cells were collected and analyzed for gene expression changes (FIG. 12). As shown in FIG. 12, xenografts demonstrated upregulation of interferons, and other cytokines/chemokines and cancer testis antigens by decitabine (DAC). Some of the biomarkers were further enhanced by the combination of decitabine (DAC) and tolinapant. The collected KARPS-299 cells were lysated, and re-expression of RIPK3 was also confirmed by western blotting, as shown in FIG. 13.

### Example 2

This is an open-label study of the safety, pharmacokinetics, pharmacodynamics, and preliminary activity of tolinapant in combination with oral decitabine/cedazuridine in subjects with relapsed/refractory peripheral T-cell lymphoma (R/R PTCL).

Phase 1 is an open-label, randomized, multi-center, 2-arm study to assess the safety of tolinapant in combination with oral decitabine/cedazuridine to determine the recommended phase 2 dose (RP2D) for the combination treatment (tolinapant plus oral decitabine/cedazuridine) in subjects with R/R PTCL. In addition, oral decitabine/cedazuridine as a single agent will be evaluated for safety and tolerated dosing in this patient population.

In the Phase 2 study, tolinapant in combination with oral decitabine/cedazuridine will be evaluated for preliminary efficacy as well as PK and PD in this patient population.

A lead-in phase will precede the start of Phase 1 to confirm that the US-approved dosing of oral decitabine/cedazuridine as a single agent is tolerated in this population. As many as 6 subjects will receive oral decitabine/cedazuridine as a single agent for Days 1-5 during a 28 day cycle. Subjects will be evaluated for myelosuppression and other dose-limiting toxicities (DLTs) and dosing adjustment will be made where applicable. Enrollment into the lead-in phase will stop if 2 or more DLTs occur in Cycle 1 and a dosing adjustment will be made for both arms of the Phase 1 study.

Phase 1 will start once the lead-in phase is complete. Subjects will be randomized (1:1) to receive tolinapant at 3 sequential dose levels (120, 150, and 180 mg/day on Days 1-7 and 15-21 of a 28-day cycle) in combination with fixed dose oral decitabine/cedazuridine (Arm A) or to a fixed dose of oral decitabine/cedazuridine as a single agent (Arm B). The starting dosing of oral decitabine/cedazuridine in both arms will be the standard FDC tablet (35 mg decitabine/100 mg cedazuridine) at a dosing regimen determined with the lead-in phase. Escalation to the next higher dose level of tolinapant will occur if applicable.

The starting dose of tolinapant in Arm A will be escalated stepwise in successive cohorts of 3 to 6 evaluable subjects at each dose level, until the RP2D is determined. The dose-escalation stage will identify the RP2D, defined as the dose that shows adequate clinical activity and safety and clinical activity. Dose-escalation/de-escalation decisions will be based on the occurrence of DLTs during the first cycle of each dose level. The tolinapant dose will not exceed 180 mg per dose and the oral decitabine/cedazuridine dosing will not extend beyond 5 days per cycle.

Certain inclusion criteria are: Men or women 18 years of age or older. Expected life expectancy of >12 weeks. Subjects must have histologically confirmed R/R PTCL (local pathology report) as defined by 2016 World Health Organization (WHO) classification. The following subtypes are eligible for the study: adult T-cell lymphoma/leukemia, extranodal natural killer (NK)/T-cell lymphoma nasal type, enteropathy-associated T-cell lymphoma, monomorphic epitheliotropic intestinal T-cell lymphoma, hepatosplenic T-cell lymphoma, subcutaneous panniculitis-like T cell lymphoma, peripheral T-cell lymphoma not otherwise specified, angioimmunoblastic T cell lymphoma, follicular T-cell lymphoma, nodal peripheral T-cell with T-follicular helper (THF) phenotype, and anaplastic large-cell lymphoma. Subjects must have documented evidence of relapsed/refractory disease.

Certain exclusion criteria include: Prior treatment with tolinapant or any hypomethylating agent. Hypersensitivity to tolinapant or oral decitabine/cedazuridine, excipients of the drug product, or other components of the study treatment regimen. Poor medical risk because of systemic diseases (e.g., uncontrolled infections) in addition to the qualifying disease under study. Life-threatening illness, significant organ system dysfunction, or other condition that, in the investigator's opinion, could compromise subject safety or the integrity of the study outcomes, or interfere with the absorption or metabolism of tolinapant. A history of, or at risk for, cardiac disease.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for treating a T-cell lymphoma in a subject in need thereof comprising administering to the subject:
tolinapant, or a pharmaceutically acceptable salt thereof;
cedazuridine, or a pharmaceutically acceptable salt thereof; and
decitabine, or a pharmaceutically acceptable salt thereof.

2. The method of claim 1, wherein the T-cell lymphoma is relapsed or refractory T-cell lymphoma.

3. The method of claim 2, wherein the T-cell lymphoma was previously treated with a hypomethylating agent.

4. The method of claim 1, wherein the T-cell lymphoma has not been previously treated with an agent for treating T-cell lymphoma.

5. The method of claim 1, wherein the T-cell lymphoma is selected from peripheral T-cell lymphomas, peripheral T-cell lymphomas not otherwise specified, angioimmunoblastic T-cell lymphoma, follicular T-cell lymphoma, nodal peripheral T-cell with T-follicular helper (THF) phenotype, adult T-cell lymphoma/leukemia, anaplastic large cell lymphoma, enteropathy-associated T-cell lymphoma, nasal NK/T-cell lymphoma, hepatosplenic T-cell lymphoma, monomorphic epitheliotropic intestinal T-cell lymphoma, subcutaneous panniculitis-like T cell lymphoma, and cutaneous (skin) T-cell lymphoma.

6. The method of claim 1, wherein the T-cell lymphoma is relapsed or refractory peripheral T-cell lymphoma (R/R PTCL).

7. The method of claim 1, wherein the T-cell lymphoma is peripheral T-cell lymphoma not otherwise specified.

8. The method of claim 1, wherein the T-cell lymphoma is angioimmunoblastic lymphoma.

9. The method of claim 1, wherein the T-cell lymphoma is anaplastic large cell lymphoma (ALCL).

10. The method of claim 1, wherein the T-cell lymphoma is hepatosplenic T-cell lymphoma.

11. The method of claim 1, wherein the T-cell lymphoma is enteropathy-associated T-cell lymphoma.

12. The method of claim 1, wherein the T-cell lymphoma is cutaneous T-cell lymphoma.

13. The method of claim 12, wherein the cutaneous T-cell lymphoma is mycosis fungoides or Sézary syndrome.

14. The method of claim 1, wherein the T-cell lymphoma is T-lymphoblastic lymphoma/leukemia or adult T-cell lymphoma/leukemia.

15. The method of claim 1, wherein the tolinapant is administered once a day for 7 consecutive days every other week of each 28-day cycle.

16. The method of claim 15, wherein the dose of tolinapant is 30 mg or 90 mg daily.

17. The method of claim 1, wherein the cedazuridine and the decitabine are administered once daily on days 1 through 5 of each 28-day cycle.

18. The method of claim 17, wherein the dose of cedazuridine is 100 mg daily and the dose of decitabine is 35 mg daily.

19. The method of claim 1, wherein the subject is not being treated with any compound that is metabolized by cytidine deaminase.

20. The method of claim 1, wherein the tolinapant, the cedazuridine, and the decitabine are administered orally.

21. A pharmaceutical composition comprising:
tolinapant, or a pharmaceutically acceptable salt thereof;
a cedazuridine, or a pharmaceutically acceptable salt thereof; and
decitabine, or a pharmaceutically acceptable salt thereof.

22.-55. (canceled)
